(19) United States
(12) Patent Application Publication
(10) Pub. No.: US 2021/0146049 A1
(43) Pub. Date: May 20, 2021

(54) NEEDLELESS INJECTION DEVICE FOR A LIQUID SOLUTION, REMOVABLE REFILL AND ASSOCIATED METHOD

(71) Applicant: ASSISTANCE PUBLIQUE HOPITAUX DE PARIS, PARIS (FR)

(72) Inventors: Marie Pierre BERLEUR, PARIS (FR); François DE TRAZEGNIES D'ITTRE, VILLEJUIF (FR); Claire LANGLET, MEAUX (FR)

(21) Appl. No.: 16/623,212

(22) PCT Filed: Jun. 19, 2018
(86) PCT No.: PCT/EP2018/066291

§ 371 Date: Dec. 16, 2019

(30) Foreign Application Priority Data
Jun. 20, 2017 (FR) 1755592

(51) Int. Cl. A61M 5/30

(52) U.S. Cl. CPC A61M 5/30; A61M 2005/3114

(57) ABSTRACT

An injection device for injecting a liquid solution, includes a power source; a deformable piezoelectric bar, the deformation of which is activated by an application of energy coming from the electrical power source; a compression injector including: a piston actuated by the deformation of the piezoelectric bar; a chamber capable of receiving a first volume of a liquid solution including an inlet for its filling and an outlet for its evacuation under the effect of the piston; and an ejection nozzle connected to the outlet of the chamber.

### TECHNICAL FIELD OF THE INVENTION

The field of the invention relates to that of needleless injection devices for expulsing a volume of a liquid solution with the objective of administering it under or within the epidermis. The field of the invention relates more specifically to injection devices using piezoelectric force. Finally, the invention relates to injection devices connected for purposes of transmission of information relative to the injected solutions.

### TECHNOLOGICAL BACKGROUND OF THE INVENTION

An important motivation exists to develop needleless solutions for injecting a medicinal solution on account of fear of needles, the risk of infectious contamination, the difficulty of self-administration or the risk of injury with the needle.

Needleless solutions making it possible to administer a medicinal solution under the epidermis include injection devices based on the use of the force of a gas or that of a spring or pyrotechnic energy.

However, certain drawbacks emerge from the use of such devices. They are usually imposing and intended for single use. They are thus expensive and behind a significant amount of waste.

Another drawback of existing solutions is that it is difficult to control the energy deployed to perform a precise injection of a given volume.

Finally, a drawback of solutions using the pyrotechnic effect is that they require the implementation of explosive cartridges that it is necessary to change.

### SUMMARY OF THE INVENTION

The invention makes it possible to resolve the aforesaid drawbacks.

According to an aspect, the invention relates to a device for injecting a liquid solution including:
An energy source;
A deformable piezoelectric bar, the deformation of which is activated by an input of energy coming from the electrical energy source;
A compression injector comprising:
   a piston actuated by the deformation of the piezoelectric bar;
   a chamber suited to receiving a first volume of a liquid solution comprising an inlet for its filling and an outlet for its evacuation under the effect of the piston;
   an ejection nozzle connected to the outlet of the chamber.

An advantage of the device of the invention is to propose a solution for injecting under the epidermis that is reliable and offers a needleless solution. This solution makes it possible to inject a predefined volume and to decrease dosage errors.

According to an embodiment, the injection device includes:
A housing able to receive a refill comprising a liquid solution and enabling its extraction to a connection element;
A valve connected to said connection element blocking or transferring said first volume of the liquid solution into the chamber.

An advantage is to make it possible to change the refill when it is empty and to conserve the device for use with another refill. This economic solution enables monitoring of injections for a patient and makes it possible to manage the quantities to administer.

According to an embodiment, the valve opens automatically under the effect of a depression of the chamber driven by a return of the piston to its initial position.

According to an embodiment, the piezoelectric bar automatically recovers its initial shape after its deformation.

According to an embodiment, the device includes a trap for closing the nozzle.

According to an embodiment, the inverse deformation of the piezoelectric bar to return to its initial shape frees the piston which returns to its initial position thereby creating a depression driving the opening of the valve.

According to an embodiment, the electrical energy source comprises a capacitor making it possible to recover the mechanical energy resulting from the contraction of the piezoelectric bar.

According to an embodiment, the injection device comprises an electronic card making it possible to acquire reference data coming from an electronic chip arranged on a refill, said electronic card enabling an exploitation of the transmitted data to generate at least one alarm.

According to an embodiment, the injection device includes a means for activating an electrical set point to generate an input of electrical energy to an associated transducer or comprised in the piezoelectric bar, the transducer generating an input of mechanical energy to deform the piezoelectric bar.

According to an embodiment, the injection device includes at least one sensor making it possible to detect a correct or incorrect positioning of the device during the activation of the electrical set point.

According to another aspect the invention relates to a refill suited to cooperating with an injection device of the invention. The refill comprises a frame having a volume intended to receive a liquid solution and of which the outer shape is intended to cooperate with a housing of a device, said refill comprising:
a first end provided with a membrane intended to be pierced by a tubular element and
a second end including a removable bottom that is displaced under the action of a depressurisation of the volume and of a sucking up of the liquid solution,
the refill comprising, moreover, an electronic label comprising at least one datum relative to the nature of the liquid solution contained in the volume.

According to an embodiment, the removable bottom is in contact with a volume of air at atmospheric pressure so as to exert pressure on the liquid solution.

According to another aspect, the invention relates to a refill suited to cooperating with an injection device of the invention. It comprises a frame having a volume intended to receive a liquid solution and of which the outer shape is intended to cooperate with a housing of a device, said refill comprising:
a first end provided with a membrane intended to be pierced by a tubular element and;
a second end including a removable bottom that is displaced under the action of a mechanical element exerting a return force.

According to another aspect, the invention relates to an injection system including an injection device and a refill suited to cooperating with said injection device.

According to another aspect, the invention relates to a method for generating an expulsion at high pressure of a liquid solution comprised in a refill from a device, the method comprising:
An activation of the deformation of a piezoelectric bar of a compression injector from a generation of an electrical control transmitted to a transducer of the piezoelectric bar;
A displacement of a piston driven by the deformation of the piezoelectric bar, said displacement of the piston driving the ejection of a liquid solution contained in a compression chamber to an outlet nozzle;
A recovery of the initial shape of the piezoelectric bar driving the return to the initial position of the piston;
A depression of the chamber of the compression injector resulting from the recoil of the piston.

According to an embodiment, the method comprises:
An insertion of a new refill in a housing of the device preceding the activation of the deformation of the piezoelectric bar;
An opening of a valve driven by the depression of the chamber and driving a sucking up of the new solution into the chamber;
An automatic closing of the valve.

According to an embodiment, the method includes a purge of the air present in the volume including the volume of the housing and those leading air to the valve.

### BRIEF DESCRIPTION OF THE FIGURES

Other characteristics and advantages of the invention will become clear from reading the detailed description that follows, with reference to the appended figures, which illustrate:

FIG. 1: an example of device according to an embodiment of the invention;

FIG. 2: an example of method according to an embodiment of the invention.

### DETAILED DESCRIPTION OF EMBODIMENTS OF THE INVENTION

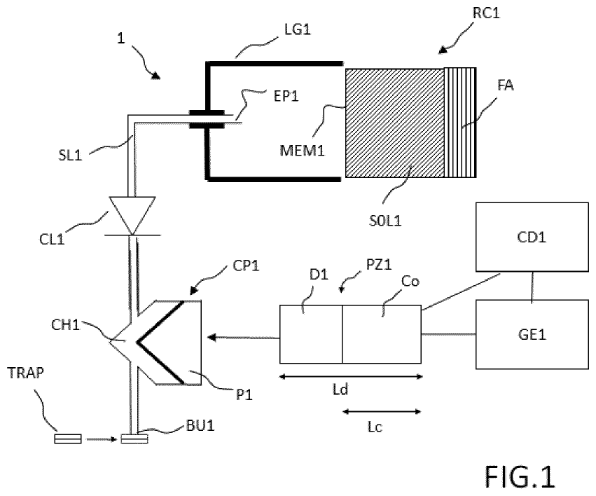

FIG. 1 represents an exemplary embodiment of the device 1 of the invention. The device 1 of the invention comprises in this example a capacitor CD1, a piezoelectric bar PZ1, a compressor CP1, a valve CL1, such as a check valve, a nozzle BU1 and a trap TR1, a housing LG1 and a refill CH1 comprising a liquid solution SOL1.

Piezoelectric Bar

The piezoelectric bar PZ1, when it is compressed, is capable of supplying a given amount of energy. Conversely, when an amount of energy is supplied to the bar, a deformation of its geometry, such as a lengthening, is generated.

An advantage of the use of a piezoelectric bar PZ1 is that it is capable of releasing a large amount of energy by its deformation when it is electrically supplied. According to an embodiment, the piezoelectric bar PZ1 includes a geometry enabling a longitudinal deformation. This deformation is controlled as a function of its dimensions and the nature of the delivered electrical set point.

According to different embodiments, the piezoelectric bar PZ1 may have a cylindrical shape or a volume with square or rectangular base. According to an embodiment, it may include a stack of piezoelectric elements.

According to an embodiment, the piezoelectric bar PZ1 may be equipped with a screw at its base to adjust the part D1 extending longitudinally. This screw may be on the one hand fixed to the piezoelectric bar and on the other hand maintained to the case. According to an exemplary embodiment, a threading makes it possible to adjust the position of the piezoelectric bar PZ1 vis-à-vis the case. In the latter situation, the screw has a rotational degree of freedom vis-à-vis the case.

Thus, due to the fact that the piezoelectric bar PZ1 is fixed on the one hand by the screw to the case and on the other hand made integral with the piston P1, the deformation of the piezoelectric bar PZ1 may be configured to evacuate a given volume of liquid according to the capacity of the chamber CH1. In other words, the screw makes it possible to adapt the capacity of the chamber CH1 to a predefined use mode. An alternative manner of configuring the deformation of the piezoelectric bar PZ1 is to drive the energy source GE1 from an electrical and/or mechanical set point.

An advantage of fixing the piezoelectric bar PZ1 to the piston P1 is to make it possible to fulfil a function of sucking up into the chamber CH1 when the piezoelectric bar PZ1 returns to its rest state.

When the screw is at its minimum, the deformation of the piezoelectric bar PZ1 generates a slight movement of the piston P1, a small fraction of the liquid solution SOL1 contained in the chamber CH1 is expulsed. Optionally, a purge may be carried out to evacuate the part of the liquid solution SOL1 remaining in the chamber CH1.

When the screw is at its maximum, the deformation of the piezoelectric bar PZ1 generates a maximum movement of the piston P1, the totality of the liquid solution SOL1 contained in the chamber CH1 is expulsed. Optionally, a purge may be carried out to clean the orifices, for example of the nozzle BU1 and optionally clean the hydraulic circuit in which the liquid solution SOL1 transits.

The deformation of the piezoelectric bar PZ1 may be engaged by an input of electrical energy. To this end, the piezoelectric bar PZ1 comprises a transducer capable of converting electrical energy into mechanical energy, known as a “piezoelectric transducer”. According to an embodiment, the activation of the piezoelectric bar PZ1 leads to an expansion of the latter in a predefined time lapse.

According to an embodiment, the geometry of the piezoelectric bar PZ1 is chosen to generate a predefined longitudinal deformation D1 and to transmit a predefined force of a thrust by contact with a piston P1. The mechanical force generated thereby is capable of pushing the piston P1 with sufficient energy in order to eject a given volume of a liquid solution comprised in a chamber CH1.

Compression Injector

The piezoelectric bar PZ1 acts on a compression injector CP1 which is also called “syringe”. According to an embodiment, the syringe is metal.

Piston

The compression injector CP1 also includes a piston P1 which is thus arranged in the device 1 of the invention to be driven by the expansion of the bar PZ1. According to an embodiment, the piston P1 and/or the chamber CH1 are composed of a chemically inactive material, such as titanium or surgical steel.

Chamber

The compression injector CP1 includes a chamber CH1 capable of receiving a volume of a liquid solution SOL1 coming from a refill RC1. According to an embodiment, the chamber CH1 is cut in the mass of the device 1, it forms a cylinder head.

According to a different embodiment, the chamber CH1 may take different shapes so as to:
receive the incoming volume of the liquid solution SOL1,
accommodate a part of the volume of the solution SOL1 in a chamber CH1 directly in contact with the piston P1 to evacuate it under the activation of the piezoelectric bar PZ1;
inject said volume through a nozzle BU1.

FIG. 1 represents an embodiment of a specific shape of the chamber CH1 making it possible to fulfil these three functions.

According to another embodiment, the chamber CH1 may have a cylindrical shape or a parallelepiped shape. According to an embodiment, the chamber has a substantially conical shape. This latter solution makes it possible to increase the surface of the piston P1 without increasing the diameter of the compression chamber CH1. This solution makes it possible, moreover, to increase the injectable volume, by reducing the loss of calorific energy generated by the compression of the liquid.

The thrust generated by the deformation of the piezoelectric bar PZ1 creates a powerful, continuous and constant pressure that drives the liquid solution SOL1 which is naturally ejected from the chamber CH1 in several milliseconds.

Nozzle

The compression injector CP1 comprises a nozzle BU1 which forms the injection tip. The nozzle BU1 may be, for example, selected at the mounting of the device and definitively fixed for later uses. Alternatively, it may be mounted removably on a support which is fixed during mounting.

According to an embodiment, the nozzle BU1 comprises a diameter comprised between 0.05 mm and 0.5 mm, for example 0.2 mm. An interest of such a diameter is to avoid the appearance of lesions near to the injection zone.

This opening makes it possible to eject the compressed solution at high pressure. The ejection pressure is comprised between 2 bars and 12 bars. According to an embodiment, the ejection pressure is adjusted to be comprised between 6 and 7 bars.

According to an embodiment, the nozzle BU1 is removeable to be changed if need be. Different diameters of nozzles BU1 may be adapted according to the use mode of the liquid solution SOL1 to expulse. The diameter of the nozzle BU1 may be chosen as a function of the deformation of the piezoelectric bar PZ1 to obtain a desired pressure at the outlet of the nozzle BU1. The outlet pressure of the nozzle BU1 makes it possible to calibrate an injection depth under the skin.

According to a first exemplary embodiment, the nozzle BU1 is made of stainless steel. According to a second example, the nozzle BU1 is made of titanium. An advantage is to have a nozzle BU1 that is not reactive chemically, that is to say chemically inert and sufficiently resistant to be fixed to the frame of the compression injector CP1. According to another embodiment, the nozzle BU1 is manufactured in the mass of the frame of the injector CP1 and forms a single-piece element therewith.

Parameterisation of the Deformation

In this manner, the device 1 of the invention may be used according to different uses, for example for sub-cutaneous or hypodermic injections. According to an embodiment, the device 1 comprises pre-adjustments making it possible to adjust the deformable part D1 of the piezoelectric bar PZ1 to obtain a desired injection range. A first alternative embodiment consists in providing the device 1 with a mechanical means such as a screw. According to a second alternative, a software means make it possible to activate a given electrical set point to generate a given deformation of the bar PZ1. According to this latter alternative, the electrical set point may be parameterizable for example in voltage to adjust the deformation of the piezoelectric bar PZ1 according to a desired use. The mechanical and software means can be combined in a same solution making it possible to define a particular embodiment of the invention.

Trap

According to an embodiment, the device 1 comprises a trap TRAP making it possible to block the outlet of the nozzle BU1 when the piston P1 is not activated. The trap TRAP is preferentially leak tight and may comprise for this purpose a seal. This makes it possible to avoid leakages of the solution SOL1. When the trap TRAP closes under the action of the recovery of the initial shape of the piezoelectric bar PZ1, it isolates the nozzle BU1.

When the trap TRAP is closed, it closes the hydraulic circuit through which the liquid solution SOL1 is ejected. The trap TRAP is closed again to contribute to creating a depression in the chamber CH1 in order that the new solution SOL1 is automatically sucked up into the chamber CH1 when the valve CL1 opens.

The trap TRAP also plays a sealing and bulwark role against the introduction of bacteria or dead skin into the hydraulic circuit of the device 1.

An advantage of the trap of the device 1 is to avoid the introduction of air, notably through the orifice of the nozzle BU1, into the hydraulic circuit in which the liquid solution SOL1 circulates.

When the piezoelectric bar PZ1 is deformed with respect to its initial shape, it activates the piston P1. The compression injector CP1 then makes it possible to compress the liquid solution SOL1 to inject it at high pressure under the epidermis of a patient. The liquid SOL1 is then evacuated along a high speed jet at the outlet of the nozzle BU1.

Energy Source

The device 1 includes an energy source GE1. According to an embodiment, the energy source GE1 is an electrical source. The piezoelectric transducer PZ1 is then capable of converting electrical energy into mechanical energy. The input mechanical energy is materialised by a deformation of the bar PZ1. According to an embodiment, the deformation is an expansion.

According to an embodiment, the device 1 may be designed such that the volume of the injection SOL1 at the outlet of the nozzle BU1 is proportional to the amplitude of the electrical voltage pulse applied to the piezoelectric bar PZ1. Consequently, the electrical intensity generated by the electrical source GE1 may be preconfigured to respond to a need at the outlet of the nozzle BU1. It may include different pre-configurations according to the embodiments, for example with refills RC1 of different sizes and a chamber CH1 of parameterizable dimensions, a viscosity of the solution or instead according to the density of the solution to inject. According to an embodiment of the invention, the electrical intensity generated is a function of an identifier of the solution to inject. According to an example, the identifier is received via a wireless interface from an electronic chip comprising the identifier. According to different possible implementations, the chip is brought into contact with a reader or it transmits information via radio.

Moreover, the shape of the piston P1, the dimensions of the chamber CH1 and the geometry of the bar PZ1 make it possible to calibrate the ratio between electrical intensity and volume expulsed at the outlet of the nozzle BU1.

The deformation of the piezoelectric bar PZ1 may be controlled by the intensity of the energy peak supplied to the piezoelectric bar PZ1. According to an embodiment, the contact surface between the bar PZ1 and that of the piston P1 is optimised to maximise the transfer of energy, they are preferentially fixed to each other. The lengthening of the piezoelectric bar PZ1 may thus be controlled in this manner.

Housing/Refill

According to an embodiment, the device 1 of the invention includes a housing LG1 to accommodate a refill RC1. The dimensions of the housing LG1 are suited to cooperating with those of a refill RC1. The refill RC1 forms a consumable in which a liquid solution SOL1 may be loaded in order to be injected under the epidermis.

An advantage is to enable a reuse of the device 1 with a plurality of refills RC1.

Use with or without Refill

In a first use, the device 1 of the invention does not include a refill CH1, for example when it is not used, or when it is sold or instead when it is stored after a treatment. Its housing LG1 is intended to receive said refill CH1, but the housing LG1 may remain empty. According to an embodiment, a plug can cover the housing LG1 in order to prevent impurities entering therein.

According to a second use, the device 1 of the invention includes the refill RC1, for example when it is used. The housing LG1 is connected to the compression chamber CP1 “syringe” through a valve CL1.

Membrane

According to an embodiment, during its introduction into the housing LG1 of the device 1, the refill RC1 is pierced so that the liquid solution SOL1 can be introduced into the device 1. According to an exemplary embodiment, the liquid SOL1 is maintained under pressure in the refill RC1 under the effect of a membrane MEM1. According to an embodiment, the membrane MEM1 is arranged at one end of the refill RC1.

During its introduction, the membrane MEM1 is perforated. To this end, the housing LG1 may be provided, according to an exemplary embodiment, with a perforation element such as a needle. The latter may be arranged in the bottom of the housing which is reached when the refill is totally inserted. According to another embodiment, the membrane is pierced by a tubular element EP1 of the housing LG1. Under the effect of the opening of a valve CL1, the liquid of the refill CH1 is sucked up and flows into the tubular element EP1 after the piercing of the membrane MEM1.

The membrane MEM1 is, for example, designed to improve the conservation of the liquid solution SOL1.

Removable Bottom

According to an embodiment, the refill RC1 includes a removable bottom FA. This removable bottom F1 is driven when the liquid solution SOL1 is sucked up outside of the housing LG1 under the effect of the opening of the valve CL1.

According to an embodiment, the removable bottom FA also enables an administration of a liquid solution SOL1 several times. According to this exemplary embodiment, the liquid solution SOL1 contained in the refill RC1 is calculated to distribute the amount expulsed in a determined number of activations of the device 1. Thus, at each activation of the device 1, after the expulsion of a volume of liquid solution SOL1, the piston P1 makes it possible to suck up a part of the liquid solution SOL1′ while leaving another part in the refill RC1. The removable bottom FA makes it possible to maintain the liquid solution SOL1 under pressure up to the new opening of the valve CL1.

The position of the removable bottom FA results from a new equilibrium position lost due to on the one hand the atmospheric pressure being exerted at its outer surface and on the other hand the depression of the hydraulic circuit during the opening of the valve CL1. The depression accompanied by the movement of the removable bottom FA making it possible to input the energy necessary to displace the liquid solution SOL1. The volume injected into the compression chamber CH1 is thus compensated by the displacement of this removable bottom FA.

According to an embodiment, the removable bottom FA is in contact with a volume of air at atmospheric pressure. This characteristic makes it possible to maintain atmospheric pressure on the removable bottom and to maintain the liquid in the cartridge with a slight pressure to facilitate the sucking up operation. To this end, the bottom of the case of the cartridge may be pierced.

According to an embodiment, a refill/a cartridge is specially designed for the upkeep of the device. It contains a cleaning liquid to rinse the device. Such a refill thus makes it possible to carry out a cleaning-decontaminant after each injection made by the device.

During cleaning, there is no need to engage a deformation of the piezoelectric bar PZ1. The cleaning cartridges then specially include a means for driving the cleaning liquid in an active manner in the device. According to an exemplary embodiment, the cartridge comprises a piston which is displaced under the effect of an element exerting a return force, such as a spring. In this embodiment, the spring is for example also comprised in the cartridge.

The piston of the cartridge may be the removable bottom FA and the spring may be arranged between the bottom of the cartridge and the removable bottom. In this case, the spring pushes the liquid up to into the chamber CH1 and then out of the nozzle BU1. The check valve is in this case opened under the effect of the liquid driven into the chamber and the trap is open.

During the insertion of a new cartridge of a liquid to inject, a priming procedure makes it possible to evacuate the residual cleaning liquid present in the device.

For example, this can be done by activating the device in an evacuation recipient, the piezoelectric bar PZ1 then deforms and drives the expulsion of the cleaning liquid still contained in the device. When the piezoelectric bar PZ1 recovers its initial shape, it disengages the piston P1 and the sucking up of a volume of liquid of the cartridge is carried out. The liquid thereby sucked up is then ready to be injected during a new operation.

Other Functions According to an embodiment, the interface between the refill RC1 and the housing LG1 is designed so that no volume of air can enter into the outlet of the housing SL1.

According to an embodiment, the housing LG1 comprises a leak tight seal and a lock making it possible to maintain the refill CH1 there within.

According to an embodiment, the refill RC1 snap fits into the housing LG1 thanks to a clip mechanism which makes it possible to validate the correct introduction of the refill CH1 in the housing LG1.

Chip Reader

According to an embodiment, the housing LG1 comprises a reader of an electronic chip. Indeed, according to an embodiment, the refill comprises an electronic chip comprising reference data. It may be an electronic chip or a radio chip, such as an RFID chip. The reader thus includes a data interface for receiving data from the chip arranged for example on the surface of the refill RC1. The reader further comprises a calculator for decoding the data received or means for transferring said data to a remote calculator. The reader identifies the data, of which notably at least one datum for identifying the refill RC1. According to different embodiments, the transmitted data include a type of refill, a solution name and/or an expiry date, a posology, etc.

Electronic Card

According to an embodiment, the device 1 comprises an electronic card and an exploitation software making it possible to exploit at least the data received from the chips of each refill RC1 inserted. The electronic card may be the card reader or a motherboard having a central function in the device 1. The electronic card comprises at least one calculator such as a microprocessor and a memory.

The software makes it possible to carry out operations on the data coming from the chips, of which operations of counting, identification, comparison, correlation and calculation. According to an embodiment, the software ensures certain functions making it possible to manage the monitoring of the injections of a patient.

For example, the number of injections carried out over a given period, the success or the failure of an injection, the referencing of the injected compositions, etc.

According to an embodiment, the device 1 comprises an interface for transmitting data or syntheses to equipment connected to a network such as a smartphone or computer or a remote data server

According to an embodiment, the electronic card and the exploitation software control the identification of a cartridge, that is to say an inserted refill RC1, and compare the identifier with reference data. The reference data may be preconfigured by the insertion of a type of acceptable liquid solution, a duration of treatment and a particular posology. When the device 1 detects that a refill CH1 is not compliant with the reference data, an alarm may be triggered.

According to an embodiment, the software calculates a number of expulsions of liquid solutions carried out for a single refill RC1 or for a plurality of refills. It is able to return a state of use of a refill RC1. Moreover, according to an embodiment, the software generates an alarm when the piezoelectric bar PZ1 has to be changed or when it can no longer ensure or guarantee a predefined deformation. According to an embodiment, the deformable part D1 of the piezoelectric bar PZ1 is adjustable and may be adjusted thanks to a screw. In this case, the software can indicate an insufficient adjustment according to the volume of solution to expulse. This function may be fulfilled thanks to an actuator returning a mechanical position to generate an electrical set point to the electronic card.

According to an embodiment, the electronic card and the software activate the generation of at least one alarm to remind, for example, a patient of the time of an injection, or when the injection has not worked correctly.

According to an embodiment, the device 1 comprises at least one sensor making it possible to control the application of the device 1 on the skin of a person. The sensor may be for example a pressure sensor or an optic sensor. In this case, the sensor may be positioned near to the injection nozzle BU1. The sensor then generates a state parameter making it possible to validate or not the activation of the electrical control triggered automatically or by a person.

According to an embodiment, a warning light or an audible signal may be generated in the event of incorrect application of the device 1 on the surface of the skin thanks to the sensor. According to an embodiment, an alarm is generated for the bearer of the device at a time lapse before the injection is carried out. An interest is to warn the bearer of the device of the imminence of the injection.

According to an embodiment, the device comprises a means for validating an injection operation after the emission of an alarm. Said means may be an electrical or mechanical control such as an operable button.

Moreover, according to an embodiment, the electronic card makes it possible to activate the electrical control of the electrical source GE1 or that of the capacitor CD1 when it plays this role. According to an embodiment, the electronic card and the software activates the opening and the closing of the trap TRAP in a synchronised manner with the activation of the piezoelectric bar PZ1.

Biometric Sensors

According to an embodiment, the device comprises at least one biometric sensor making it possible to record physiological data of the patient. According to an exemplary embodiment, a temperature sensor makes it possible to record the temperature of the skin. The data may be recorded in a memory of the electronic card or the electronic chip.

According to an embodiment, the device comprises a heart rate sensor making it possible to measure the pulse. The collected data may be recorded in a memory of the electronic card.

According to an embodiment, the device comprises an arterial pressure sensor. The pressure data collected may be recorded in a memory of the electronic card.

The recorded data may be time-stamped and associated with a cartridge identifier of a liquid administered in the skin of a patient for example a time lapse before or a time lapse after a recording.

Valve

According to an embodiment, the device 1 of the invention comprises a valve CL1. According to an example, the valve CL1 is of check valve type. It makes it possible on the one hand to maintain its closed position during the phase of compression of the compression chamber CH1 and on the other hand to allow the liquid SOL1 to pass when the chamber CH1 of the compression injector CP1 is depressurised following the recoil of the piston P1. Its role is to manage the fillings of the chamber CH1 after a preceding use.

When the piezoelectric bar PZ1 contracts once again following its lengthening, the piston P1 again moves back to recover its equilibrium position in the compression injector CP1. This leads to a lowering of the pressure in the chamber CH1 and the opening of the valve CL1. The valve CL1 being open, the next volume of liquid SOL2 enters into the chamber RC1 for the next ejection.

Indeed, the depression applied to the refill CH1 during the opening of the valve CL1 causes a sucking up of the liquid solution SOL1 and a movement of the removable bottom FA. The volume of liquid escaping is compensated by the displacement of the moveable bottom FA of the refill RC1 of the solution SOL1.

Energy Source and Capacitor

According to an embodiment, the device 1 of the invention includes an electrical energy source GE1. According to an embodiment, an electric battery (or a fuel cell) can deliver, from a transformer, an electrical set point in voltage or in current making it possible to activate the piezoelectric bar PZ1 thanks to the piezoelectric transducer.

According to another embodiment, a capacitor CD1 may be used to collect and to recover a part of the dissipated energy of the piezoelectric bar PZ1. It may be associated with an electrical source GE1 or be autonomous to deliver an electrical set point to the piezoelectric bar PZ1 to deform it. When the piezoelectric bar PZ1 recovers its initial shape, the mechanical energy may be recovered and converted into electrical energy which is supplied to the capacitor. Thus, the device 1 of the invention makes it possible to recover a part of the dissipated energy to extend its autonomy. According to an embodiment, the capacitor includes an inlet interface for receiving an incoming current coming either from a recovery of mechanical/electrical energy of the device 1, or an internal source, or an external source.

Geometry

According to an embodiment, the device is integrated in a frame of around 50 cm³. According to an exemplary embodiment, the dimensions correspond to those of a nano-computer of 53 mm×53 mm perimeter and 16 mm thickness. The thickness is dimensioned in this case by a dimension of the piezoelectric bar PZ1.

According to an exemplary embodiment, the piezoelectric bar PZ1 includes a length of 29 mm and a diameter of 16 mm.

An advantage of the device 1 of the invention is that the deformation of the piezoelectric bar PZ1 is reliable and enables a large number of deformation cycles by lengthening and by contraction. Thus, the volume of each liquid solution SOL1 is fully expulsed thanks to the constancy of the deformation of the piezoelectric bar.

According to an embodiment, the device 1 may take the form of a patch, an armband, a bracelet or a watch.

Method of the Invention

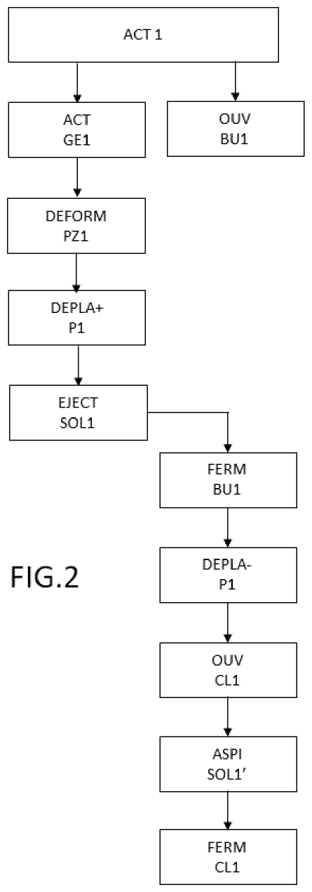

According to the different embodiments of the method of the invention, one or more of the following steps are carried out.
An activation of the device 1, noted ACT1 in FIG. 2, to achieve an expulsion of a volume of liquid solution. The activation of the device 1 is achieved from a control such as a button or a tactile interface. According to an embodiment, a confirmation request is generated and certain control parameters are verified. For example, among these parameters, there may be a parameter of a positioning sensor of the device, a parameter for controlling the time, a parameter for controlling the nature of the liquid solution, etc.
The activation of the device 1 includes an activation of an electrical set point ACT_GE1 coming from an electrical source making it possible to activate the deformation of the piezoelectric bar PZ1: DEFORM_PZ1. Moreover, according to an embodiment, the activation of the device ACT1 comprises an activation of the opening of the trap OUV_BU1 freeing the orifice of the nozzle BU1 to enable the high pressure ejection of the liquid solution SOL1.
A movement of a piston, noted DEPLA+_P1, is driven by the deformation DEFORM_PZ1 of the piezoelectric bar PZ1.
An ejection EJECT_SOL1 of the liquid solution SOL1 contained in a compression chamber CH1 to the outlet nozzle BU1.
A closing FERM_BU1 of the trap TRAP after the ejection of the solution SOL1.
A recovery of the initial shape of the piezoelectric bar PZ1.
A recoil movement DEPLA−_P1 of the piston P1 to return to its initial position.
A depressurisation of the chamber CH1 of the compression injector CP1 resulting from the recoil of the piston P1.
An opening OUV_CLAP of the valve CL1 driven by the depressurisation of the chamber CH1.
A sucking up ASPI_SOL1′ of a part or a new solution SOL1′ of a refill RC 1 introduced beforehand into the housing LG1, said new solution SOL1 being introduced automatically into the chamber CH1 thanks to the opening of the valve CL1.
An automatic closing FERM_CL1 of the valve CL1.

According to an embodiment, at each insertion of a new refill CH1, a purge of the air present in the circuit is carried out.

1. An injection device for injecting a liquid solution, comprising:
an energy source;
a deformable piezoelectric bar, a deformation of which is activated by an input of energy coming from the electrical energy source;
a compression injector comprising:
a piston actuated by the deformation of the piezoelectric bar;
a chamber configured to receive a first volume of a liquid solution comprising an inlet for its filling and an outlet for its evacuation under the effect of the piston;
an ejection nozzle connected to the outlet of the chamber.
a housing capable of receiving a refill comprising a liquid solution and enabling its extraction to a connection element;
an electronic card configured to acquire reference data coming from an electronic chip arranged on a refill, said electronic card enabling an exploitation of the transmitted data to generate at least one alarm.

2. The injection device according to claim 1, further comprising:
a check valve connected to said connection element blocking or transferring said first volume of the liquid solution into the chamber.

3. The injection device according to claim 2, wherein the check valve is arranged to open automatically under the effect of a depression of the chamber driven by a return of the piston to its initial position.

4. The injection device according to claim 2, wherein the piezoelectric bar is configured to recover its initial shape automatically after its deformation.

5. The injection device according to claim 1, further comprising a trap for closing the nozzle.

6. The injection device according to claim 1, wherein the piezoelectric bar is configured so that its inverse deformation to return to its initial shape frees the piston which returns to its initial position, thereby creating a depression driving the opening of the valve.

7. The injection device according to claim 1, wherein the electrical energy source comprises a capacitor configured to recover the mechanical energy resulting from the contraction of the piezoelectric bar and/or to store electrical energy to deliver an electrical set point to the piezoelectric bar in order to cause the mechanical deformation of said piezoelectric bar.

8. The injection device according to claim 1, further comprising a system for activating an electrical set point to generate an input of electrical energy to an associated transducer or comprised in the piezoelectric bar, the transducer generating an input of mechanical energy to deform the piezoelectric bar.

9. The injection device according to claim 8, further comprising at least one sensor configured to detect a correct or incorrect positioning of the device during the activation of the electrical set point.

10. A refill configured to cooperate with a device of claim 1, comprising a frame having a volume intended to receive a liquid solution and of which an outer shape is configured to cooperate with a housing of the device, said refill comprising:
a first end provided with a membrane intended to be pierced by a tubular element and;
a second end including a removable bottom that is displaced under the action of a depressurisation of the volume and a sucking up of the liquid solution,
the refill comprising, moreover, an electronic label comprising at least one datum relative to the nature of the liquid solution contained in the volume.

11. The refill according to claim 10, wherein the removable bottom is configured to be in contact with a volume of air at atmospheric pressure in such a way as to exert pressure on the liquid solution.

12. A refill configured to cooperate with a device of claim 1, comprising a frame having a volume intended to receive a liquid solution and of which an outer shape is configured to cooperate with a housing of the device, said refill comprising:
a first end provided with a membrane intended to be pierced by a tubular element and;
a second end including a removable bottom being displaced under the action of a mechanical element exerting a return force.

13. An injection system comprising an injection device according to claim 1 and a refill configured to cooperate with said injection device.
